(19) United States
(12) Patent Application Publication
(10) Pub. No.: US 2005/0196362 A1
(43) Pub. Date: Sep. 8, 2005

(54) Personal care compositions containing highly branched primary alcohol component

(76) Inventors: Jan Elsa Eugenia Carty, Sugar Land, TX (US); Carolyn Ann Burnley, Houston, TX (US); Kenneth Klein, Fairfield, NJ (US)

Correspondence Address: SHELL OIL COMPANY, P O BOX 2463, HOUSTON, TX, 772522463, US

(21) Appl. No.: 11/069,793

(22) Filed: Mar. 1, 2005

Related U.S. Application Data
(60) Provisional application No. 60/549,333, filed on Mar. 2, 2004.
(60) Provisional application No. 60/577,633, filed on Jun. 7, 2004.

(51) Int. Cl. A61K 7/42

(52) U.S. Cl. 424/59

(57) ABSTRACT

A personal care composition for topical application to the skin or hair comprising (a) a branched primary alcohol component, having from 8 to 36 carbon atoms and an average number of branches per molecule of from 0.7 to 3.0, said branching comprising methyl and ethyl branches, and said branched primary alcohol component optionally comprising up to 3 moles of alkylene oxide per mole of alcohol; (b) one or more sunscreens; and (c) a cosmetically-acceptable vehicle. The personal care compositions of the invention provide excellent sunscreen protection, stability, viscosity and rheology characteristics, together with emolliency, application and skin feel benefits.

### REFERENCE TO PRIOR APPLICATIONS

This application claims the benefit of U.S. Provisional application Ser. No. 60/549,333, filed Mar. 2, 2004, and U.S. Provisional application Ser. No. 60/577,633, filed Jun. 7, 2004, the entire disclosures of which are herein incorporated by reference.

### FIELD OF THE INVENTION

The present invention relates to a personal care composition for topical application to the skin or hair comprising a highly branched primary alcohol component and at least one sunscreen.

### BACKGROUND OF THE INVENTION

It is well known in the art that light radiation of wavelengths from 280 nm to 400 nm is harmful to the skin. In particular, UV-B radiation (of wavelengths 290 nm to 320 nm) is known to cause erythema and burning of the skin and therefore it is desirable to screen the skin from UV-B radiation. It is also known that UV-A radiation (of wavelengths 320 to 400 nm) can cause a loss in the elasticity of skin and the appearance of wrinkles. Therefore, it is also desirable to screen the skin from UV-A radiation.

A wide variety of cosmetic compositions suitable for screening the skin from UV-A and/or UV-B radiation are known in the art. These photoprotective/sunscreen compositions are often oil-in-water emulsions which contain, in various concentrations, one or more UV-A and/or UV-B sunscreens. These sunscreens may be UV-absorbing organic screening agents or inorganic pigments which scatter and/or reflect UV radiation, as well as mixtures thereof. The types and amounts of sunscreen compounds are selected as a function of the desired sun protection factor (SPF). SPF is expressed mathematically by the ratio of the irradiation time required to attain the erythema-forming threshold with the UV screening agent to the time required to attain the erythema-forming threshold in the absence of UV screening agent.

There exists an increasing demand for personal care products having higher SPFS. High SPFs can sometimes be attained by incorporating higher levels of sunscreens. However, this is not always feasible as high sunscreen levels can lead to products which have undesirably high viscosities, an increased possibility of irritation and increased formula cost. Additionally, adding more sunscreen sometimes actually lowers the SPF due to agglomeration as a result of a polarity mismatch between the sunscreen and the solvent used to solubilise the sunscreen at high sunscreen concentrations.

Various solubilising/dispersing agents are known in the art for solubilising/dispersing sunscreen compounds in personal care compositions. However there is still a need to provide sunscreen compositions which can contain high levels of sunscreen compounds while maintaining product viscosity at an acceptable level.

It has now surprisingly been found that the use of a particular branched primary alcohol component having from 0.7 to 3.0 branches per molecule may be used effectively for solubilising/dispersing sunscreen compounds so as to provide a personal care composition containing high levels of sunscreen(s) (and thereby a high SPF) together with an acceptable product viscosity.

U.S. Pat. No. 5,849,960 (Shell Oil Company) discloses a branched primary alcohol composition having 8 to 36 carbon atoms which contains an average number of branches per molecule of at least 0.7, said branching comprising methyl and ethyl branching. These alcohols may subsequently be converted to anionic or nonionic detergents or general surfactants by sulfonation or ethoxylation, respectively, of the alcohol. The detergents produced exhibit useful properties such as high biodegradability and high cold water detergency. No disclosure is provided in U.S. Pat. No. 5,849,960 of the use of these branched alcohols in personal care compositions.

WO99/18929, WO99/18928 and WO97/39089 (The Procter and Gamble Company) disclose personal cleansing compositions comprising mid-chain branched surfactants. The mid-chain branched surfactants are manufactured from mid-chain branched alcohols. The formulations therein however do not contain mid-chain branched alcohols per se, only the corresponding surfactants. In addition, these documents are concerned with cleansing compositions having relatively high levels of surfactant ingredients.

### SUMMARY OF THE INVENTION

According to the present invention there is provided a personal care composition for topical application to the skin or hair comprising
(a) a branched primary alcohol component, having from 8 to 36 carbon atoms and an average number of branches per molecule of from 0.7 to 3.0, said branching comprising methyl and ethyl branches, and said branched primary alcohol component optionally comprising up to 3 moles of alkylene oxide per mole of alcohol;
(b) at least one sunscreen; and
(c) a cosmetically-acceptable vehicle.

According to a further aspect of the present invention there is provided the use of a branched alcohol component for dispersing organic and/or inorganic sunscreens in a personal care composition, wherein the branched primary alcohol component has from 8 to 36 carbon atoms and an average number of branches per molecule of from 0.7 to 3.0, said branching comprising methyl and ethyl branches.

Said branched alcohol component may be used effectively to solubilise/disperse sunscreen compounds so as to provide personal care compositions containing high levels of sunscreen(s) (and therefore high SPFs) and which have acceptable product viscosities.

The personal care compositions of the present invention also have excellent stability and rheology characteristics, together with excellent emolliency, application and skin feel benefits.

### BRIEF DESCRIPTION OF THE DRAWINGS

FIG. 1 is a line graph showing the relationship between the amount of Z-cote (zinc oxide commercially available from BASF Corporation) in grams and the viscosity of a dispersion of Z-cote in an indicated solubiliser/dispersant, the dispersion being prepared according to Example 2.

FIG. 2 is a line graph showing the relationship between the amount of Z-cote HP1 (hydrophobically-modified zinc oxide commercially available from BASF Corporation) in grams and the viscosity of a dispersion of Z-cote HP1 in an indicated solubiliser/dispersant, the dispersion being prepared according to Example 2.

FIG. 3 is a line graph showing the relationship between the amount of Eusolex T2000 (titanium dioxide commercially available from Merck & Co.) in grams and the viscosity of a dispersion of Eusolex T2000 in an indicated solubiliser/dispersant, the dispersion being prepared according to Example 2.

FIG. 4 is a bar chart showing the amount of Z-cote in grams required to provide a dispersion having a viscosity of 10,000 cps, the dispersion being a dispersion of Z-cote in an indicated solubilizer/dispersant prepared according to Example 2

FIG. 5 is a bar chart showing the amount of Z-cote HP1 in grams required to provide a dispersion having a viscosity of 10,000 cps, the dispersion being a dispersion of Z-cote HP1 in an indicated solubilizer/dispersant prepared according to Example 2

FIG. 6 is a bar chart showing the amount of Eusolex T2000 in grams required to provide a dispersion having a viscosity of 10,000 cps, the dispersion being a dispersion of Eusolex T2000 in an indicated solubilizer/dispersant prepared according to Example 2.

FIG. 7 shows the minimum weight of pigment required to target 10,000 cps sunscreen viscosity when titanium dioxide and zinc oxide are used and the Mod OXO Mono-Methyl C1617 Branched Alcohol.

FIG. 8 shows the minimum weight of modified pigment required to target 10,000 cps sunscreen viscosity when Z-COTE HP1 is used and the Mod OXO Mono-Methyl C1617 Branched Alcohol.

### DETAILED DESCRIPTION OF THE INVENTION

All percentages and ratios used herein are by weight of the total personal care composition, unless otherwise specified.

All publications cited herein are incorporated by reference in their entirety, unless otherwise indicated.

The term “cosmetically-acceptable”, as used herein, means that the compositions, or components thereof, are suitable for use in contact with human skin or hair without undue toxicity, incompatibility, instability, allergic response, and the like.

The term “safe and effective amount” as used herein means an amount of a compound, component, or composition sufficient to significantly induce a positive benefit, e.g. a sun protection benefit, a skin feel benefit or a skin appearance benefit, but low enough to avoid serious side effects, i.e. to provide a reasonable benefit to risk ratio, within the scope of sound medical judgement.

The elements of the personal care compositions of the invention are described in more detail below.

Branched Primary Alcohol Component

A first essential component of the personal care compositions herein is a branched primary alcohol component having from 8 to 36 carbon atoms and an average number of branches per molecule of from 0.7 to 3.0, said branching comprising methyl and ethyl branching. In addition, the branched primary alcohol component may optionally comprise up to 3 moles of alkylene oxide per mole of alcohol.

The branched primary alcohol component is particularly useful herein for effectively solubilising/dispersing one or more sunscreen compounds. Personal care compositions containing said branched alcohol component may therefore contain high levels of sunscreen (and therefore a high SPF) while maintaining an acceptable product viscosity.

The personal care compositions of the present invention comprise a safe and effective amount of the branched primary alcohol component described herein. Suitably the personal care compositions of the present invention comprise from 0.01 to 30%, preferably from 0.1 to 20%, more preferably from 0.5% to 15% and especially from 1% to about 10% by weight of the branched primary alcohol component.

As used herein, the phrase “average number of branches per molecule chain” refers to the average number of branches per alcohol molecule, as measured by 13C Nuclear Magnetic Resonance (13C NMR) as discussed below, or optionally ¹H Proton NMR. The average number of carbon atoms in the chain is determined by gas chromatography with a mass selective detector.

Various references will be made throughout this specification and the claims to the percentage of branching at a given carbon position, the percentage of branching based on types of branches, average number of branches, and percentage of quaternary atoms. These amounts are to be measured and determined by using a combination of the following three 13C-NMR techniques. (1) The first is the standard inverse gated technique using a 45-degree tip 13C pulse and 10 s recycle delay (an organic free radical relaxation agent is added to the solution of the branched alcohol in deuterated chloroform to ensure quantitative results). (2) The second is a J-Modulated Spin Echo NMR technique (JMSE) using a 1/J delay of 8 ms (J is the 125 Hz coupling constant between carbon and proton for these aliphatic alcohols). This sequence distinguishes carbons with an odd number of protons from those bearing an even number of protons, i.e. CH₃/CH vs CH₂/Cq (Cq refers to a quaternary carbon). (3) The third is the JMSE NMR “quat-only” technique using a 1/2J delay of 4 ms which yields a spectrum that contains signals from quaternary carbons only.

The JSME NMR quat only technique for detecting quaternary carbon atoms is sensitive enough to detect the presence of as little at 0.3 atom % of quaternary carbon atoms. As an optional further step, if one desires to confirm a conclusion reached from the results of a quat only JSME NMR spectrum, one may also run a DEPT-135 NMR sequence. We have found that the DEPT-135 NMR sequence is very helpful in differentiating true quaternary carbons from break-through protonated carbons. This is due to the fact that the DEPT-135 sequence produces the “opposite” spectrum to that of the JMSE “quat-only” experiment. Whereas the latter nulls all signals except for quaternary carbons, the DEPT-135 nulls exclusively quaternary carbons. The combination of the two spectra is therefore very useful in spotting non quaternary carbons in the JMSE “quat-only” spectrum. When referring to the presence or absence of quaternary carbon atoms throughout this specification, however, we mean that the given amount or absence of the quaternary carbon is as measured by the quat only JSME NMR method. If one optionally desires to confirm the results, then also using the DEPT-135 technique to confirm the presence and amount of a quaternary carbon.

The primary alcohol component used in the invention contains an average chain length per molecule ranging from about 8 to about 36 carbon atoms, preferably from about 11 to about 21 carbon atoms. The number of carbon atoms includes carbon atoms along the chain backbone as well as branching carbons, but does not include carbon atoms in alkylene oxide groups.

Preferably, at least 75 wt %, more preferably, at least 90 wt. % of the molecules in the primary alcohol component have chain lengths ranging from 11 to 21, yet more preferably from 14 to 18 carbon atoms.

The average number of branches per molecule is at least 0.7, as defined and determined above. Preferred alcohol components are those having an average number of branches of from 0.7 to 3.0, preferably from 1.0 to 3.0. Particularly preferred alcohol components are those having an average number of branches of at least 1.5, in particular ranging from 1.5 to about 2.3, especially from 1.7 to 2.1.

In a preferred embodiment of the invention the primary alcohol component has less than 0.5 atom % of Cq's as measured by a quat-only JMSE modified 13C-NMR having a detection limit of 0.3 atom % or better, and preferably contains no Cq's as measured by this NMR technique. For reasons not yet clearly understood, it is believed that the presence of Cq's on an alcohol molecule prevents the biodegradation by biological organisms. Alcohols containing as little as 1 atom % of Cq's have been been found to biodegrade at failure rates.

In a preferred embodiment of the invention, less than 5%, or more preferably less than 3%, of the alcohol molecules in the primary alcohol component are linear alcohols. The efficient reduction in the number of linear alcohols to such a small amount in the composition results from introducing branching on an olefin feedstock either by a skeletal isomerization or a dimerisation technique using efficient catalysts as described further below, rather than introducing branching by methods such as acid catalyzed oligomerization of propylene molecules, or zeolite catalyzed oligomerization techniques. The percentage of molecules which are linear may be determined by gas chromatography.

Skeletal Isomerization

In a preferred embodiment herein, the branching is introduced by skeletal isomerization.

When the branching has been achieved by skeletal isomerization, the primary alcohol component used herein may be characterized by the NMR technique as having from 5 to 25% branching on the C2 carbon position, relative to the hydroxyl carbon atom. In a more preferred embodiment, from 10 to 20% of the number of branches are at the C2 position, as determined by the NMR technique. The primary alcohol component also generally has from 10% to 50% of the number of branches on the C3 position, more typically from 15% to 30% on the C3 position, also as determined by the NMR technique. When coupled with the number of branches seen at the C2 position, the primary alcohol component contains significant amount of branching at the C2 and C3 carbon positions.

Not only does the primary alcohol component used in the present invention have a significant number of branches at the C2 and C3 positions, but we have also seen by the NMR technique that many of the primary alcohol components have at least 5% of isopropyl terminal type of branching, meaning methyl branches at the second to last carbon position in the backbone relative to the hydroxylcarbon. We have even seen at least 10% of terminal isopropyl types of branches in the primary alcohol component, typically in the range of 10% to 20%. In typical hydroformylated olefins of the NEODOL series commercially available from The Shell Chemical Company, less than 1%, and usually 0.0%, of the branches are terminal isopropyl branches. By skeletally isomerizing the olefin according to the invention, however, the primary alcohol component contains a high percentage of terminal isopropyl branches relative to the total number of branches.

Considering the combined number of branches occurring at the C2, C3, and isopropyl positions, there are embodiments of the invention where at least 20%, more preferably at least 30%, of the branches are concentrated at these positions. The scope of the invention, however, includes branching occurring across the length of the carbon backbone.

The types of branching found in the primary alcohol composition of the invention varies from methyl, ethyl, propyl, and butyl or higher.

In a preferred embodiment of the invention, the total number of methyl branches number at least 40%, even at least 50%, of the total number of branches, as measured by the NMR technique described above. This percentage includes the overall number of methyl branches seen by the NMR technique described above within the C1 to the C3 carbon positions relative to the hydroxyl group, and the terminal isopropyl type of methyl branches.

The primary alcohol component herein contains a significant increase in the number of ethyl branches over those seen on NEODOL alcohols such as NEODOL 45. The number of ethyl branches may range from 5% to 30%, most typically from 10% to 20%, based on the overall types of branching that the NMR method detects. Thus, the skeletal isomerization of the olefins produces both methyl and ethyl branches. Thus, the types of catalysts one may use to perform skeletal isomerization are not restricted to those which will produce only methyl branches. The presence of a variety of branching types is believed to enhance a good overall balance of properties.

The olefins used in the olefin feed for skeletal isomerization are at least C₇ mono-olefins: In a preferred range, the olefin feed comprises C₇ to C₃₅ mono-olefins. Olefins in the C₁₁ to C₁₉ range are considered most preferred for use herein, to produce primary alcohol components in the C₁₂ to C₂₀ range.

In general, the olefins in the olefin feed composition are predominantly linear. Attempting to process a predominantly branched olefin feed, containing quaternary carbon atoms or extremely high branch lengths, would require separation methods after passing the olefin stream across the catalyst bed to separate these species from the desired branched olefins. While the olefin feed may contain some branched olefins, the olefin feed processed for skeletal isomerization preferably contains greater than about 50 percent, more preferably greater than about 70 percent, and most preferably greater than about 80 mole percent or more of linear olefin molecules.

The olefin feed generally does not consist of 100% olefins within the specified carbon number range, as such purity is not commercially available. The olefin feed is usually a distribution of mono-olefins having different carbon lengths, with at least 50 wt. % of the olefins being within the stated carbon chain range or digit, however specified. Preferably, the olefin feed will contain greater than 70 wt. %, more preferably about 80 wt. % or more of mono-olefins in a specified carbon number range (e.g., C₇ to C₉, C₁₀ to C₁₂, C₁₁ to C₁₅, C₁₂ to C₁₃, C₁₅ to C₁₈, etc.), the remainder of the product being olefin of other carbon number or carbon structure, diolefins, paraffins, aromatics, and other impurities resulting from the synthesis process. The location of the double bond is not limited. The olefin feed composition may comprise α-olefins, internal olefins, or a mixture thereof.

Chevron Alpha Olefin product series (trademark of and sold by Chevron Chemical Co.), manufactures predominantly linear olefins by the cracking of paraffin wax. Commercial olefin products manufactured by ethylene oligomerization are marketed in the United States by Shell Chemical Company under the trademark NEODENE and by Ethyl Corporation as Ethyl Alpha-Olefins. Specific procedures for preparing suitable linear olefins from ethylene are described in U.S. Pat. Nos. 3,676,523, 3,686,351, 3,737,475, 3,825,615 and 4,020,121. While most of such olefin products are comprised largely of alpha-olefins, higher linear internal olefins are also commercially produced, for example, by the chlorination-dehydro-chlorination of paraffins, by paraffin dehydrogenation, and by isomerization of alpha-olefins. Linear internal olefin products in the C₈ to C₂₂ range are marketed by Shell Chemical Company and by Liquichemica Company.

Skeletal isomerization of linear olefins may be carried out by any known means. Preferably herein, skeletal isomerization is carried out using the process of U.S. Pat. No. 5,849,960, with use of a catalytic isomerization furnace. Preferably an isomerization feed as hereinbefore defined is contacted with an isomerization catalyst which is effective for skeletal isomerising a linear olefin composition into an olefin composition having an average number of branches per molecule chain of at least 0.7. More preferably the catalyst comprises a zeolite having at least one channel with a crystallographic free channel diameter ranging from greater than 4.2 Angstrom and less than 7 Angstrom, measured at room temperature, with essentially no channel present which has a free channel diameter which is greater than 7 Angstrom.

Suitable zeolites are described in U.S. Pat. No. 5,510,306, the contents of which are incorporated herein by reference, and are described in the Atlas of Zeolite Structure Types by W. M. Meier and D. H. Olson. Preferred catalysts include ferrierite, AlPO-31, SAPO-11, SAPO-31, SAPO-41, FU-9, NU-10, NU-23, ZSM-12, ZSM-22, ZSM-23, ZSM-35, ZSM-48, ZSM-50, ZSM-57, SUZ-4A, MeAPO-11, MeAPO-31, MeAPO-41, MeAPSO-11, MeAPSO-31, and MeAPSO-41, MeAPSO-46, ELAPO-11, ELAPO-31, ELAPO-41, ELAPSO-11, ELAPSO-31, and ELAPSO-41, laumontite, cancrinite, offretite, hydrogen form of stilbite, the magnesium or calcium form of mordenite and partheite, and their isotypic structures. Combinations of zeolites may also be used herein. These combinations may include pellets of mixed zeolites and stacked bed arrangements of catalyst such as, for example, ZSM-22 and/or ZSM-23 over ferrierite, ferrierite over ZSM-22 and/or ZSM-23, and ZSM-22 over ZSM-23. The stacked catalysts may be of the same shape and/or size or of different shape and/or size such as 1/8 inch trilobes over 1/32 inch cylinders for example. Alternatively natural zeolites may be altered by ion exchange processes to remove or substitute the alkali or alkaline earth metal, thereby introducing larger channel sizes or reducing larger channel sizes. Such zeolites include natural and synthetic ferrierite (may be orthorhombic or monoclinic), Sr-D, FU-9 (EP B-55,529), ISI-6 (U.S. Pat. No. 4,578,259), NU-23 (E.P.A.-103,981), ZSM-35 (U.S. Pat. No. 4,016,245) and ZSM-38 (U.S. Pat. No. 4,375,573). Most preferably the catalyst is ferrierite.

The skeletal isomerization catalyst is suitably combined with a refractory oxide as binding material in known manner, for example natural clays, such as bentonite, montmorillonite, attapulgite, and kaolin; alumina; silica; silica-alumina; hydrated alumina; titania; zirconia and mixtures thereof. More preferred binders are aluminas, such as pseudoboehmite, gamma and bayerite aluminas. These binders are readily available commercially and are used to manufacture alumina-based catalysts.

The weight ratio of zeolite to binder material suitably ranges from about 10:90 to about 99.5:0.5, preferably from about 75:25 to about 99:1, more preferably from about 80:20 to about 98:2 and most preferably from about 85:15 to about 95:5 (anhydrous basis).

Preferably, the skeletal isomerization catalyst is also prepared with at least one acid selected from mono-carboxylic acids and inorganic acids and at least one organic acid with at least two carboxylic acid groups (“polycarboxylic acid”). Suitable acids include those disclosed in U.S. Pat. No. 5,849,960.

Optionally, coke oxidation promoting metals may be incorporated into the instant catalysts to promote the oxidation of coke in the presence of oxygen at a temperature greater than about 250° C. Suitable coke oxidation promoting materials include those disclosed in U.S. Pat. No. 5,849,960.

In a preferred method, the instant catalysts may be prepared by mixing a mixture of at least one zeolite as herein defined, alumina-containing binder, water, at least one monocarboxylic acid or inorganic acid and at least one polycarboxylic acid in a vessel or a container, forming a pellet of the mixed mixture and calcining the pellets at elevated temperatures. Preparation methods of the catalyst are described in U.S. Pat. No. 5,849,960.

High conversion, high selectivity, and high yields are attained by the process described herein.

The present skeletal isomerization process may be operated at a wide range of conditions. Preferably skeletal isomerization is conducted at elevated temperature in the range 200° C. to 500° C., more preferably 250 to 350° C., and at pressure ranging from 0.1 atmospheres (10 kPa) to 10 atmospheres (1 MPa), more preferably from 0.5 to 5 atmospheres (50 to 500 kPa). Olefin weight hour space velocity (WHSV) may range from 0.1 to 100 per hour. Preferably, the WHSV is between 0.5 to 50, more preferably between 1 and 40, most preferably between 2 and 30 per hour. At lower WHSV's, it is possible to operate at lower temperatures while achieving high yields of skeletally isomerized branched olefins. At higher WHSV's, the temperature is generally increased in order to maintain the desired conversion and selectivity to the skeletally isomerized branched olefins. Further, optimal selectivities are generally achieved at lower olefin partial pressures mentioned above. For this reason, it is often advantageous to dilute the feed stream with a diluent gas such as nitrogen or hydrogen. Although reducing the olefin partial pressure with a diluent may be beneficial to improve the selectivity of the process, it is not necessary to dilute the olefin stream with a diluent.

If a diluent is used, the molar ratio of olefin to diluent may range from 0.01:1 to 100:1, and is generally within the range of 0.1:1 to 5:1.

Although in the present invention, skeletal isomerization is preferred, branching may also be achieved by dimerization.

Broadly speaking, a primary alcohol component is obtained by dimerizing an olefin feed comprising C₆-C₁₀ linear olefins in the presence of a dimerization catalyst under dimerization conditions to obtain C12-C20 olefins. Details of suitable dimerisation processes, including process conditions, olefin feed and suitable catalysts, are to be found in U.S. Pat. No. 5,780,694.

Hydroformylation

The branched, skeletally isomerized or dimerized, olefins are subsequently converted to a primary alcohol component, for example, by hydroformylation. In hydroformylation, the skeletally isomerized olefins are converted to alkanols by reaction with carbon monoxide and hydrogen according to the Oxo process. Most commonly used is the “modified Oxo process”, using a phosphine, phosphite, arsine or pyridine ligand modified cobalt or rhodium catalyst, as described in U.S. Pat. Nos. 3,231,621; 3,239, 566; 3,239,569; 3,239,570; 3,239,571; 3,420,898; 3,440,291; 3,448,158; 3,448,157; 3,496,203; and 3,496,204; 3,501,515; and 3,527,818. Methods of production are also described in Kirk Othmer, “Encyclopedia of Chemical Technology” 3rd Ed. vol 16, pages 637-653; “Monohydric Alcohols: Manufacture, Applications and Chemistry”, E. J. Wickson, Ed. Am. Chem. Soc. 1981.

Hydroformylation is a term used in the art to denote the reaction of an olefin with CO and H2 to produce an aldehyde/alcohol which has one more carbon atom than the reactant olefin. Frequently, in the art, the term hydroformylation is utilized to cover the aldehyde and the reduction to the alcohol step in total, i.e., hydroformylation refers to the production of alcohols from olefins via carbonylation and an aldehyde reduction process. As used herein, hydroformylation refers to the ultimate production of alcohols.

Illustrative catalysts include, but are not necessarily limited to, cobalt hydrocarbonyl catalysts and metal-phosphine ligand catalysts comprising metals, including but not limited to, palladium, cobalt and rhodium. The choice of catalysts deter-mines the various reaction conditions imposed. These conditions may vary widely, depending upon the particular catalysts. For example, temperatures may range from about room temperatures to 300° C. When cobalt carbonyl catalysts are used, which are also the ones typically used, temperatures will range from 150° to 250° C. One of ordinary skill in the art, by referring to the above-cited references, or any of the well-known literature on oxo alcohols can readily determine those conditions of temperature and pressure that will be needed to hydroformylate the isomerized or dimerized olefins.

Typical reaction conditions, however, are moderate. Temperatures in the range of 125° C. to 200° C. are recommended. Reaction pressures in the range of 2170 to 10440 kPa are typical, but lower or higher pressures may be selected. Ratios of catalyst to olefin ranging from 1:1000 to 1:1 are suitable. The ratio of hydrogen to carbon monoxide may vary widely, but is usually in the range of 1 to 10, preferably about 2 moles of hydrogen to one mole of carbon monoxide to favor the alcohol product.

The hydroformylation process may be carried out in the presence of an inert solvent, although it is not necessary. A variety of solvents may be applied such as ketones, e.g. acetone, methyl ethyl ketone, methyl iso-butyl ketone, acetophenone and cyclohexanone; aromatic compounds such as benzene, toluene and the xylenes; halogenated aromatic compounds such as chlorobenzene and orthodichlorobenzene; halogenated paraffinic hydrocarbons such as methylene chloride and carbon tetrachloride; paraffins such as hexane, heptane, methylcyclohexane and isooctane and nitriles such as benzonitrile and acetonitrile.

With respect to the catalyst ligand, mention may be made of tertiary organo phosphines, such as trialkyl phosphines, triamyl phosphine, trihexyl phosphine, dimethyl ethyl phosphine, diamylethyl phosphine, tricyclopentyl(or hexyl) phosphine, diphenyl butyl phosphine, diphenyl benzyl phosphine, triethoxy phosphine, butyl diethyoxy phosphine, triphenyl phosphine, dimethyl phenyl phosphine, methyl diphenyl phosphine, dimethyl propyl phosphine, the tritolyl phosphines and the corresponding arsines and stibines.

Included as bidentate-type ligands are tetramethyl diphosphinoethane, tetramethyl diphosphinopropane, tetraethyl diphosphinoethane, tetrabutyl diphosphinoethane, dimethyl diethyl diphosphinoethane, tetraphenyl diphosphinoethane, tetraperfluorophenyl diphosphinoethane, tetraphenyl diphosphinopropane, tetraphenyl diphosphinobutane, dimethyl diphenyl diphosphinoethane, diethyl diphenyl diphosphinopropane and tetratrolyl diphosphinoethane.

Examples of other suitable ligands are the phosphabicyclohydrocarbons, such as 9-hydrocarbyl-9-phosphabicyclononane in which the smallest P-containing ring contains at least 5 carbon atoms. Some examples include 9-aryl-9-phosphabicyclo[4.2.1]nonane, (di)alkyl-9-aryl-9-phosphabicyclo[4.2.1]nonane, 9-alkyl-9-phosphabi-cyclo[4.2.1]nonane, 9-cycloalkyl-9-phosphabicyclo-[4.2.1]nonane, 9-cycloalkenyl-9-phosphabicyclo-[4.2.1]nonane, and their [3.3.1] and [3.2.1] counter-parts, as well as their triene counterparts.

Ethoxylation

As mentioned above, the branched primary alcohol component may optionally comprise up to 3 moles of alkylene oxide per mole of alcohol. The upper limit on the number of moles of alkylene oxide reflects the fact that the primary alcohol component should not act as a surfactant in the compositions herein.

Suitable oxyalkylated alcohols may be prepared by adding to the alcohol or mixture of alcohols to be oxyalkylated a calculated amount, e.g., from about 0.1% by weight to about 0.6% by weight, preferably from about 0.1% by weight to about 0.4% by weight, based on total alcohol, of a strong base, typically an alkali metal or alkaline earth metal hydroxide such as sodium hydroxide or potassium hydroxide, which serves as a catalyst for oxyalkylation. The resulting mixture is dried, as by vapour phase removal of any water present, and an amount of alkylene oxide calculated to provide from about 1 mole to about 3 moles of alkylene oxide per mole of alcohol is then introduced and the resulting mixture is allowed to react until the alkylene oxide is consumed, the course of the reaction being followed by the decrease in reaction pressure.

Further details of suitable oxyalkylation processes including process conditions may be found in U.S. Pat. No. 6,150,322.

Suitable alkylene oxides for use herein include ethylene oxide, propylene oxide and butylene oxide, and mixtures thereof, preferably ethylene oxide.

Suncreen(s)

The personal care compositions herein also comprise one or more sunscreens.

The one or more sunscreens for use herein may be selected from organic sunscreens, inorganic sunscreens and mixtures thereof.

Any inorganic or organic sunscreen suitable for use in a personal care composition may be used herein. The level of sunscreen used depends on the required level of Sun Protection Factor, “SPF”. In order to provide a high level of protection from the sun, the SPF of the personal care composition should be at least 15, more preferably at least 20.

Suitable inorganic sunscreens for use herein include, but are not necessarily limited to, cerium oxides, chromium oxides, cobalt oxides, iron oxides, titanium dioxide, zinc oxide and zirconium oxide and mixtures thereof.

The inorganic sunscreens used herein may or may not be hydrophobically-modified, for example, silicone-treated. In preferred embodiments herein, the inorganic sunscreens are not hydrophobically-modified.

Preferred inorganic sunscreens for use herein are selected from titanium dioxide, zinc oxide and mixtures thereof.

Examples of inorganic sunscreens suitable for use herein include zinc oxide commercially available from BASF Corporation under the tradename Z-cote, hydrophobically-modified zinc oxide commercially available from BASF Corporation under the tradename Z-cote HP1 and titanium dioxide commercially available from Merck & Co., under the tradename Eusolex T2000.

The inorganic sunscreens, when present in the personal care compositions herein, are used in a safe and effective amount, preferably from 2% to 25% by weight, more preferably from 3% to 15% by weight, of composition.

Suitable organic sunscreens for use herein include those having UVA absorbing properties, those having UVB absorbing properties and mixtures thereof. Examples of suitable organic sunscreens include those listed in U.S. Pat. No. 6,436,377.

Suitable organic sunscreens for use herein include p-aminobenzoic acid derivatives, anthranilates, benzophenones, camphor derivatives, cinnamic derivatives, dibenzoyl methanes, β,β-diphenylacrylate derivatives, salicylic derivatives, triazine derivatives, benzimidazole derivatives, bis-benzoazolyl derivatives, methylene bis(hydroxyphenylbenzotriazole) compounds, the sunscreen polymers and silicones, or mixtures thereof.

Examples of suitable organic sunscreens for use herein include 4-(1,1-dimethylethyl)-4′methoxydibenzoylmethane, which is also known as butyl methoxydibenzoylmethane or Avobenzone, commercially available under the tradename Parsol 1789 from Givaudan Roure S. A., Switzerland; benzophenone-8 (also known as dioxybenzone); benzophenone-3 (also known as oxybenzone); benzophenone-4 (also known as sulisobenzone); 2-ethylhexyl-2-cyano-3,3-diphenylacrylate (commonly known as octocrylene), 2-phenyl-benzimidazole-5-sulphonic acid (PBSA) (also known as ensulizole); 2-ethylhexyl-p-methoxycinnamate (also known as octyl-p-methoxycinnamate or octinoxate); TEA salicylate (also known as trolamine salicylate); ethylhexyl salicylate (also known as octisalate); ethylhexyl p-aminobenzoate (also known as homosalate); aminobenzoic acid (PABA), menthyl anthranilate (also known as meradimate); ethylhexyldimethyl PABA (also known as Padimate O); methylbenzylidine camphor; ethylhexyl triazone (commercially available under the tradename Uvinul T150 from BASF Aktiengesellschaft, Fine Chemicals Division, 67056 Ludwigshafen, Germany); diethylamino hydroxybenzoyl hexyl benzoate (commercially available from BASF under the tradename Uvinul A Plus); methylene bis-benzotriazolyl tetramethylbutylphenol (commercially available from Ciba Speciality Chemicals under the tradename Tinasorb M); and bis-ethylhexyloxyphenol methoxyphenyl triazine (commercially available from Ciba Speciality Chemicals under the tradename Tinasorb S); and mixtures thereof.

Particularly preferred organic sunscreens for use herein are selected from 2-ethylhexyl-p-methoxycinnamate, ethylhexyl salicylate, benzophenone-3, octocrylene and butyl methoxydibenzoylmethane, and mixtures thereof.

The organic sunscreens, when present in the personal care compositions herein, are used in a safe and effective amount, preferably from 2% to 25%, more preferably from 4% to 20%, by weight of composition.

Cosmetically-Acceptable Vehicle

The personal care compositions herein also comprise a cosmetically-acceptable vehicle in addition to the primary branched alcohol component. The cosmetically-acceptable vehicle is generally present in a safe and effective amount, preferably from 1% to 99.99%, more preferably from about 20% to about 99%, especially from about 60% to about 90%. The cosmetically-acceptable vehicle may contain a variety of components suitable for rendering such compositions cosmetically, aesthetically or otherwise, acceptable or to provide them with additional usage benefits. The components of the cosmetically-acceptable vehicle should be physically and chemically compatible with the primary branched alcohol component and should not unduly impair the stability, efficacy or other benefits associated with the personal care compositions of the invention.

Suitable ingredients for inclusion in the cosmetically-acceptable vehicle are well known to those skilled in the art. These include, but are not limited to, emollients, oil absorbents, antimicrobial agents, binders, buffering agents, denaturants, cosmetic astringents, film formers, humectants, surfactants, emulsifiers, oils such as vegetable oils, mineral oil and silicone oils, opacifying agents, perfumes, colouring agents, pigments, skin soothing and healing agents, preservatives, propellants, skin penetration enhancers, solvents, suspending agents, emulsifiers, cleansing agents, thickening agents, solubilising agents, waxes, inorganic sunblocks, sunless tanning agents, antioxidants and/or free radical scavengers, chelating agents, suspending agents, sunless tanning agents, antioxidants and/or radical scavengers, anti-acne agents, anti-dandruff agents, anti-inflammatory agents, exfolients/desquamation agents, organic hydroxy acids, vitamins, natural extracts, inorganic particulates such as silica and boron nitride, deodorants and antiperspirants.

Non limiting examples of such materials are described in Harry's Cosmeticology, 7^{th} Edition., Harry & Wilkinson (Hill Publishers, London 1982); in The Chemistry and Manufacture of Cosmetics, 2^{nd}. Edition., deNavarre (Van Nostrand 1962-1965); and in the Handbook of Cosmetic Science and Technology, 1^{st} Edition., Knowlton & Pearce (Elsevier 1993); CTFA International Cosmetic Ingredient Dictionary and Handbook, 7th Edition, volume 2, edited by Wenniger and McEwen (The Cosmetic, Toiletry, and Fragrance Association, Inc., Washington, D.C., 1997); and WO01/89466.

Preferred compositions may have an apparent viscosity of from 500 cps to about 300,000 cps, preferably from 1,000 cps to about 100,000 cps, measured using a Brookfield DVII RV viscometer, spindle TD, at 5 rpm, 25° C. and ambient pressure. The viscosity may vary depending on whether the composition is a cream or lotion.

Compositions of the present invention are preferably aqueous, and more preferably are in the form of an emulsion, such as an oil-in-water or water-in-oil emulsion. For example, in the case of an oil-in-water emulsion a hydrophobic phase containing an oily material is dispersed within an aqueous phase. Oil-in-water emulsions typically comprise from 1% to 50%, preferably from 10% to 40% by weight of the dispersed hydrophobic phase and from 20% to about 90%, more preferably from 40% to about 75% by weight of the continuous aqueous phase. The emulsion may also comprise a gel network, such as described in G. M. Eccelston, Application of Emulsion Stability Theories to Mobile and Semisolid O/W Emulsions, Cosmetic & Toiletries, Vol. 101, November 1996, pp. 73-92.

The compositions of the invention will preferably be formulated to have a pH of from about 4.5 to about 9, more preferably from about 5 to about 8.5.

The compositions herein may be formulated into a wide variety of product forms such as are known in the art and may be used for a wide variety of purposes. Suitable product forms include, but are not limited to, lotions, creams, gels, sticks, sprays, ointments, pastes and mousses.

In preferred embodiments herein the personal care compositions are formulated as non-cleansing formulations, preferably comprising 5% or less, more preferably 3% of less, by weight, of surfactant.

Any surfactant known for use in personal care compositions may be used herein, provided that the selected agent is chemically and physically compatible with other ingredients in the composition. Suitable surfactants for use in the compositions herein include nonionic, anionic, amphoteric, zwitterionic and cationic surfactants, such as those described in WO01/89466.

Preferred cosmetically-acceptable vehicles herein contain a hydrophilic diluent, typically at a level of 60% to 99% by weight of composition. Suitable hydrophilic diluents include water, low molecular weight monohydric alcohols, glycols and polyols, including propylene glycol, polypropylene glycol, glycerol, butylene glycol, sorbitol esters, ethanol, isopropanol, ethoxylated ethers, propoxylated ethers and mixtures thereof. A preferred diluent is water.

The cosmetically-acceptable vehicle herein may contain an emulsifier to help disperse and suspend the discontinuous phase within the continuous aqueous phase. An example of a suitable emulsifier is PEG-30 dihydroxystearate commercially available from Uniqema Americas and a mixture of glyceryl Stearate and PEG-100 stearate commercially available under the tradename Lipomulse 165 from Lipo Chemicals, Inc., 207 19^{th} Avenue, Paterson, N.J. 07504, USA.

Preferred compositions herein comprise emollient materials, in addition to the primary branched alcohol component which itself has emolliency properties. Emollients are materials which lubricate the skin, increase the softness and smoothness of the skin, prevent or relieve dryness, and/or protect the skin. Emollients are typically oily or waxy materials which are water-immiscible. In an oil-in-water emulsion, emollients therefore generally form part of the disperse oil phase. Suitable emollients are described in Sagarin, Cosmetics, Science and Technology, 2^{nd} Edition, Vol. 1, pp. 32-43 (1972) and in WO01/89466.

Examples of preferred emollients include those disclosed in WO01/89466 such as straight and branched chain hydrocarbons having from 7 to 40 carbon atoms, such as dodecane, squalane, cholesterol, isohexadecane and the C₇-C₄₀ isoparaffins, C₁-C₃₀ alcohol esters of C₁-C₃₀ carboxylic acids and of C₂-C₃₀ dicarboxylic acids such as isononyl isononanoate, isopropyl myristate, myristyl propionate, isopropyl stearate, isopropyl isostearate, methyl isostearate, behenyl behenate, octyl palmitate, dioctyl maleate, diisopropyl adipate, and diisopropyl dilinoleate, C₁-C₃₀ mono- and poly-esters of sugars and related materials such as those disclosed in WO01/89466; and vegetable oils and hydrogenated vegetable oils including safflower oil, castor oil, coconut oil, cottonseed oil, palm kernal oil, palm oil, peanut oil, soybean oil, rapeseed oil, linseed oil, rice bran oil, pine oil, sesame oil, sunflower seed oil, partially and fully hydrogenated oils of the above, and mixtures thereof.

Preferred compositions herein contain silicone-based ingredients such as volatile or non-volatile organopolysiloxane oils. Preferred for use herein are organopolysiloxanes selected from polyalkylsiloxanes, alkyl substituted dimethicones, dimethiconols, polyalkylaryl siloxanes and cyclomethicones, preferably polyalkylsiloxanes and cyclomethicones. Also useful herein are silicone-based emulisifers such as dimethicone copolyols, an example of which is cetyl dimethicone copolyol, supplied by Goldschmidt under the tradename Abil EM90.

The compositions herein preferably comprise a thickening agent such as those described in WO01/89466. Suitable thickening agents include carboxylic acid polymers, crosslinked polyacrylates, polyacrylamides, xanthan gum, cellulose derivatives, and mixtures thereof. Examples of suitable thickening agents include the Carbopol series of materials commercially available from Noveon Hilton Davis, Inc., 2235 Langdon Farm Road, Cincinnati, Ohio 45237, USA and cetyl hydroxymethyl cellulose supplied by Hercules Aqualon under the tradename Natrosol 250 HR CS.

Preferred compositions herein comprise a humectant at a level of about 5% to about 30% by weight. Preferred humectants include, but are not limited to, glycerine, polyoxyalkylene glycol, urea, D or DL panthenol and alkylene glycols such as propylene glycol or butylene glycol.

The compositions herein may comprise a long chain alcohol in addition to the branched primary alcohol component. Suitable long chain alcohols may be selected from linear or branched, saturated or unsaturated alcohols having 30 an average number of carbon atoms in the range of from 8 to 36.

Examples of naturally derived long chain alcohols include the fatty alcohols cetyl alcohol, stearyl alcohol and behenyl alcohol.

Other suitable long chain alcohols include those commercially available from The Shell Chemical Company under the tradename NEODOL. Examples of NEODOL alcohols include NEODOL 23, NEODOL 91, NEODOL 1, NEODOL 45 and NEODOL 25. All of these alcohols are predominantly linear alcohols.

Other suitable alcohols include alcohols of the SAFOL series such as SAFOL 23, alcohols of the LIAL series such LIAL 123, and alcohols of the ALFONIC series, all of which are commercially available from Cognis.

Also suitable for use herein are the so-called “Guerbet” alcohols, for example, EUTANOL G16, commercially available from Sasol.

The compositions herein may be prepared according to procedures usually used in cosmetics and that are well known and understood by those skilled in the art.

The present invention will now be illustrated by the following Examples, which should not be regarded as limiting the scope of the present invention in any way, by reference to the accompanying drawings.

### Example 1

This example will demonstrate the manufacture of a skeletally isomerized C₁₆ olefin, subsequently converted to a skeletally isomerized C₁₇ primary alcohol component. The manufacturing process for this Example is as described in Example 1 of U.S. Pat. No. 5,849,960, but is reproduced here for convenience.

1 Litre of NEODENE 16 olefin, a C₁₆ linear α-olefin commercially available from Shell Chemical Company, was first dried and purified through alumina. The olefin was then passed through a tube furnace at about 250° C. set at a feed rate of about 1.0 ml/minute and using a nitrogen pad flowing at about 91 ml/minute. Working from the top, the tube furnace was loaded with glass wool, then 10 ml of silicon carbide, then the catalyst, followed by 5 ml of silicon carbide, and more glass wool at the bottom. The volume of the tube furnace was 66 ml. The reactor tube furnace had three temperature zones, with a multi-point thermocouple inserted into the tube reactor and positioned such that the temperature above, below and at three different places in the catalyst bed could be monitored. The reactor was inverted and installed in the furnace. All three zones, including the catalyst zone, were kept at about 250° C. during the reaction and the pressure was maintained in the reactor at 114 kPa. The amount of catalyst used was 23.1 g, or 53 ml by volume. The type of catalyst used to structurally isomerize the NEODENE 16 olefin was a 1.59 mm extruded and calcined H-ferrierite containing 100 ppm palladium metal.

This catalyst was prepared in accordance with example C of U.S. Pat. No. 5,510,306, reproduced in part herein for convenience. An ammonium-ferrierite having a molar silica to alumina ratio of 62:1, a surface area of 369 square meters per gram (P/Po=0.03), a soda content of 480 ppm and n-hexane sorption capacity of 7.3 g per 100 g of zeolite was used as the starting zeolite. The catalyst components were mulled using a Lancaster mix muller. The mulled catalyst material was extruded using an 25.4 mm or a 57.2 mm Bonnot pin barrel extruder.

The catalyst was prepared using 1 wt % acetic acid and 1 wt % citric acid. The Lancaster mix muller was loaded with 645 grams of ammonium-ferrierite (5.4% Loss on Ignition) and 91 grams of CATAPAL D alumina (LOI of 25.7%). The alumina was blended with the ferrierite for 5 minutes during which time 152 millilitres of deionized water was added. A mixture of 6.8 grams glacial acetic acid, 7.0 grams of citric acid and 152 milliliters of deionized water was added slowly to the muller in order to peptize the alumina. The mixture was mulled for 10 minutes. 0.20 grams of tetra-ammine palladium nitrate in 153 grams of deionized water were then added slowly as the mixture was mulled for a period of 5 additional minutes. Ten grams of METHOCEL F4M hydroxypropyl methylcellulose was added and the zeolite/alumina mixture was mulled for 15 additional minutes. The extrusion mix had an LOI of 43.5%. The 90:10 zeolite/alumina mixture was transferred to the 2.25 inch Bonnot extruder and extruded using a die plate with 1.59 mm holes.

The moist extrudates were tray dried in an oven heated to 150° C. for 2 hours, and then increased to 175° C. for 4 hours. After drying, the extrudates were longs-broken manually. The extrudates were calcined in flowing air at 500° C. for two hours.

The olefin was passed through the reactor furnace over a 5 hour period. Samples of 36.99 g and 185.38 g were collected at about the 1 and 5 hour point, and combined for a total of about 222 g. A portion of this sample was then vacuum distilled at 0.533 kPa to obtain a predominate amount of the C₁₆ skeletally isomerized olefin by collecting distillate cuts boiling at 160° C. in the pot and 85° C. at the head, and 182° C. in the pot and 75° C. at the head.

A 90 gram sample of the 110.93 grams of the skeletally isomerized olefin was then hydroformlyated using the modified oxo process. 90 grams of the skeletally isomerized olefin was reacted with hydrogen and carbon monoxide in about a 1.7:1 molar ratio in the presence of a phosphine modified cobalt catalyst at a temperature of up to about 185° C. and a pressure of about 7684 kPa for 4.5 hours in a nitrogen purged 300 cc autoclave. After completion of the reaction, the product was cooled to 60° C.

40 grams of the hydroformylated product was poured into a 100 ml flask and vacuum distilled for 4 hours at 0.533 kPa with temperature increases from a start temperature of 89° C. to a finish temperature of 165° C. Distillate cuts of 20.14 g and 4.12 g were taken at 155° C. and 165° C., respectively, and combined in a 100 ml flask.

To the distillate cuts in the flask was added 0.2 g of sodium borohydride, stirred, and heated up to 90° C. over an 8 hour period to deactivate the hydroformylation catalyst and stabilize the alcohols. The distilled alcohol was washed with 90° C. water three times, dried with sodium sulfate, and filtered into a 100 ml flask. The alcohol was then vacuum distilled for a further hour to distil off any remaining water.

The primary branched alcohol component prepared in accordance with Example 1 was subsequently tested for amount, type, and location of branching using the JSME NMR method described herein. For a determination of quaternary carbon atoms, the quat only JSME NMR technique described herein was used.

Results were as follows: The average number of carbon atoms in the primary alcohol component prepared according to Example 1 was found to be 17, with an average of 1.6 branches per chain. 67.9% of branching occurred at the C4 position and further (relative to the hydroxylcarbon), with 21% of branching at C3, 4% of methyl branching at C2, 1.2% of ethyl branching at C2, 5.9% of propyl branching and longer at C2, 41.7% propyl branching and longer, 16.3% ethyl branching and longer, 42% methyl branching, 0% isopropyl terminal branching, <1% linear alcohol.

Finally, in spite of the high number of branches per molecule chain, no quaternary carbon atoms were detected by the modified NMR JSME method. This would suggest that the compounds of Example 1 should readily biodegrade.

### Example 2

100 grams of the branched primary alcohol component prepared according to Example 1 was mixed with 15 grams of Z-cote (zinc oxide commercially available from BASF Corporation, Nutrition and Cosmetics, 3000 Continental Drive North, Mt Olive, N.J. 07828, USA). The branched primary alcohol component and the Z-cote were mixed for 15 minutes at 5000 rpm using a Silverson mixer to form a dispersion of zinc oxide in the alcohol component. The dispersion was allowed to equilibrate to room temperature (25° C.), and then viscosity measurements were taken using a Brookfield (RTM) RVT Viscometer set at 20 rpm. Additional Z-cote was then added to the branched alcohol component in incremental amounts of 15 g. Viscosity measurements were taken after mixing each incremental amount of Z-cote with the branched alcohol component for 15 minutes and after allowing the resulting dispersion to return to room temperature. When the viscosity of the dispersion reached or exceeded 10,000 cps no further additions of Z-cote were made and no further viscosity measurements were taken. A target viscosity of 10,000 cps was selected since a dispersion having a viscosity of greater than 10,000 cps is not preferred for use in a sunscreen composition.

For comparison, the same experiments were repeated using other commonly known sunscreen solubilisers/dispersants in place of the branched alcohol component prepared according to Example 1, namely, isohexadecane (commercially available from Presperse Inc. under the tradename Permethyl 101A), isododecane commercially available under the tradename Permethyl 99A from Presperse Inc.), octyldodecyl neopentanoate ester (commercially available from Bernel Chemical Co. under the tradename Elefac I-205, caprylic/capric triglyceride (commercially available from Lipo Chemicals Inc., under the tradename Liponate GC, C12-C15 alkyl benzoate ester (commercially available from Finetex Inc., under the tradename Finsolv TN), cyclomethicone (commercially available from Dow Corning Corporation under the tradename DC345), Mineral Oil and dioctylsebacate. Unless otherwise indicated, 100 g of the solubilizer/dispersant was used in place of the branched alcohol component prepared according to Example 1.

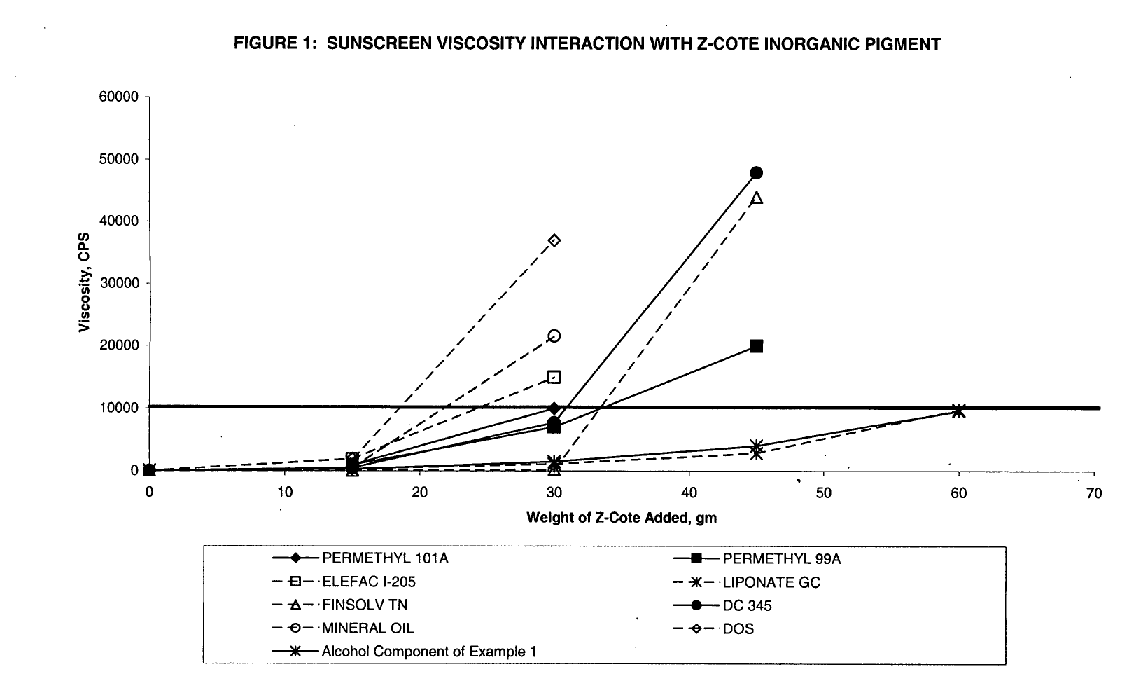

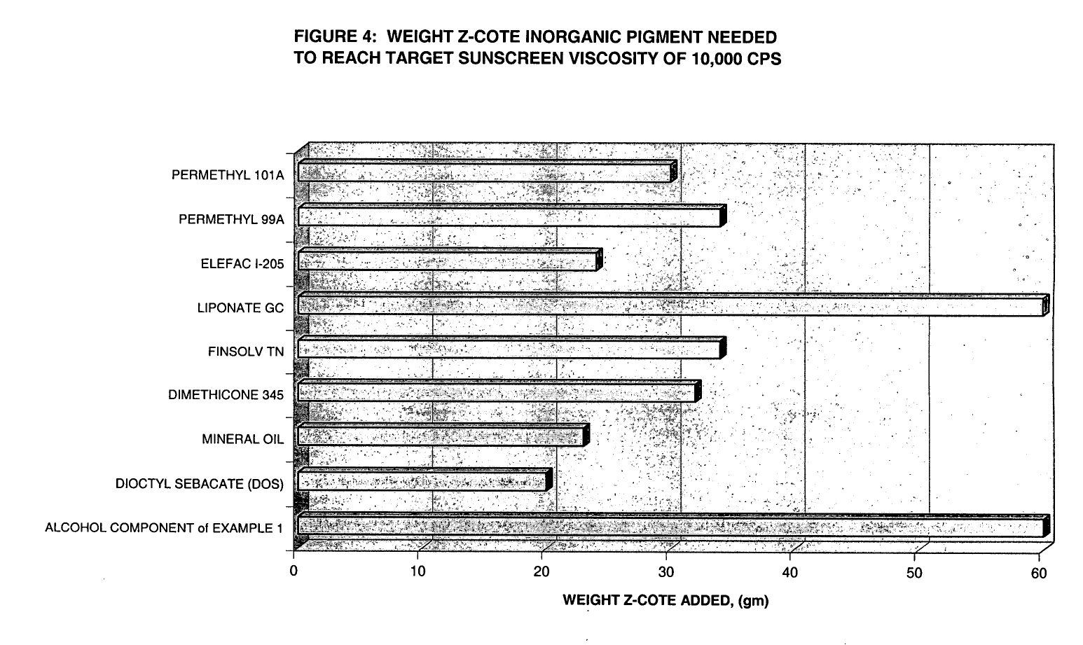

FIG. 1 shows the relationship between the amount of Z-cote in grams and the viscosity of a dispersion of Z-cote in an indicated solubiliser/dispersant, the dispersion being prepared according to the method described in Example 2. From FIG. 1, the amount of Z-cote needed to provide a dispersion having a viscosity of 10,000 cps may be determined for each solubilizer/dispersant. Table 1 below and FIG. 4 show the amount of Z-cote required to give a dispersion (in the indicated solubilizer) having a viscosity of 10,000 cps.

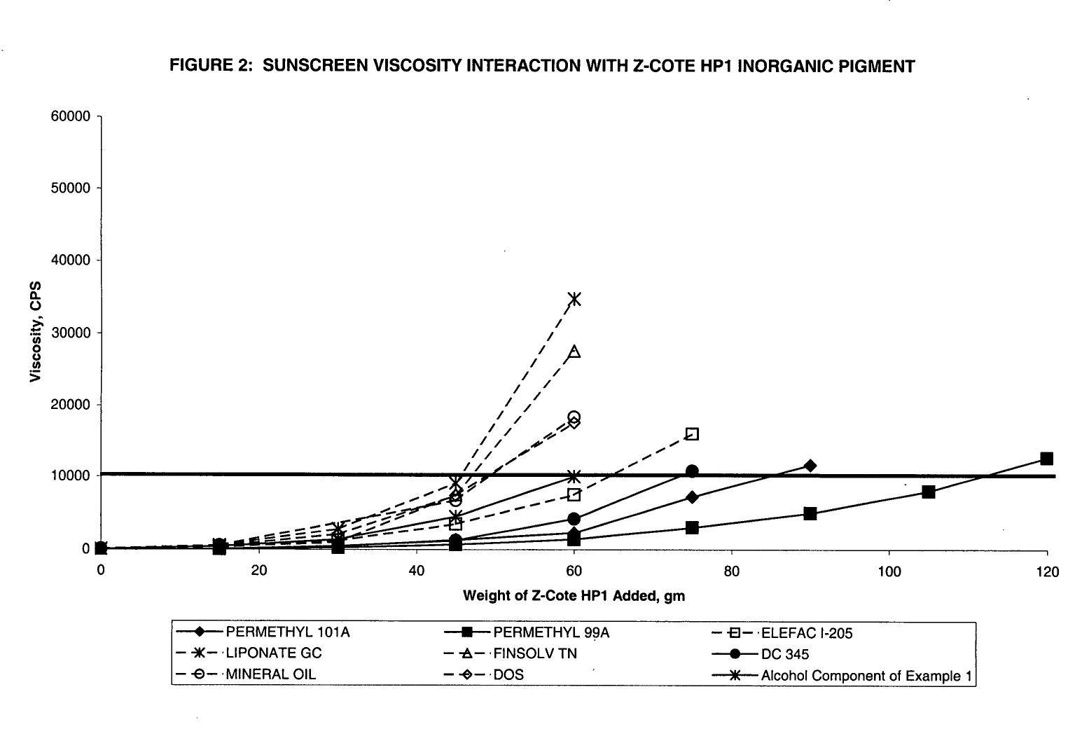

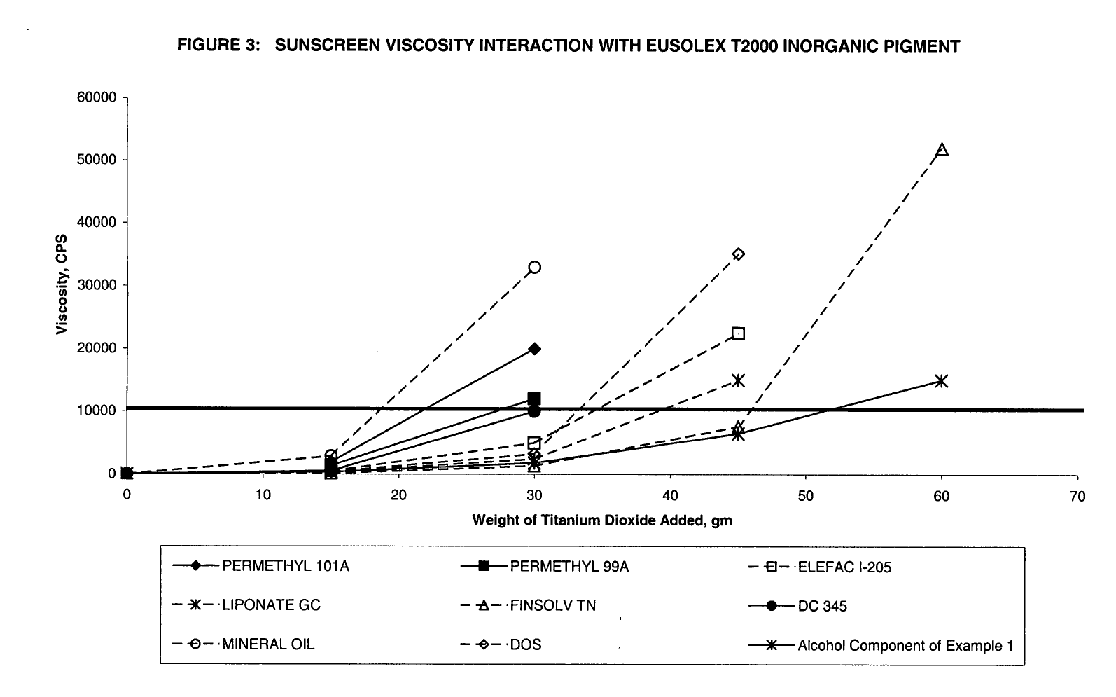

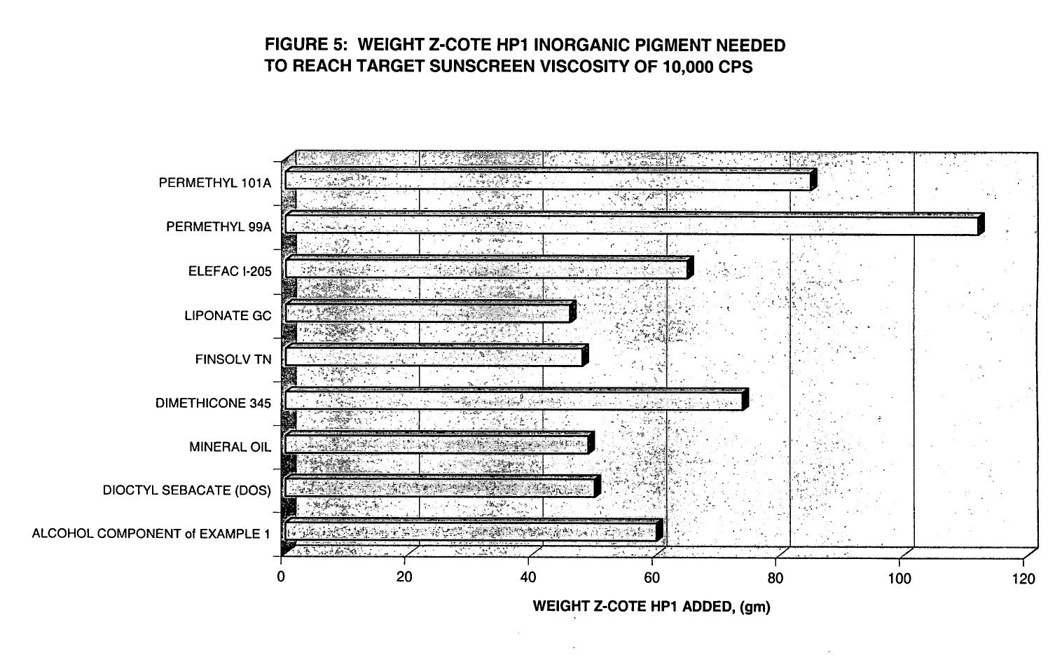

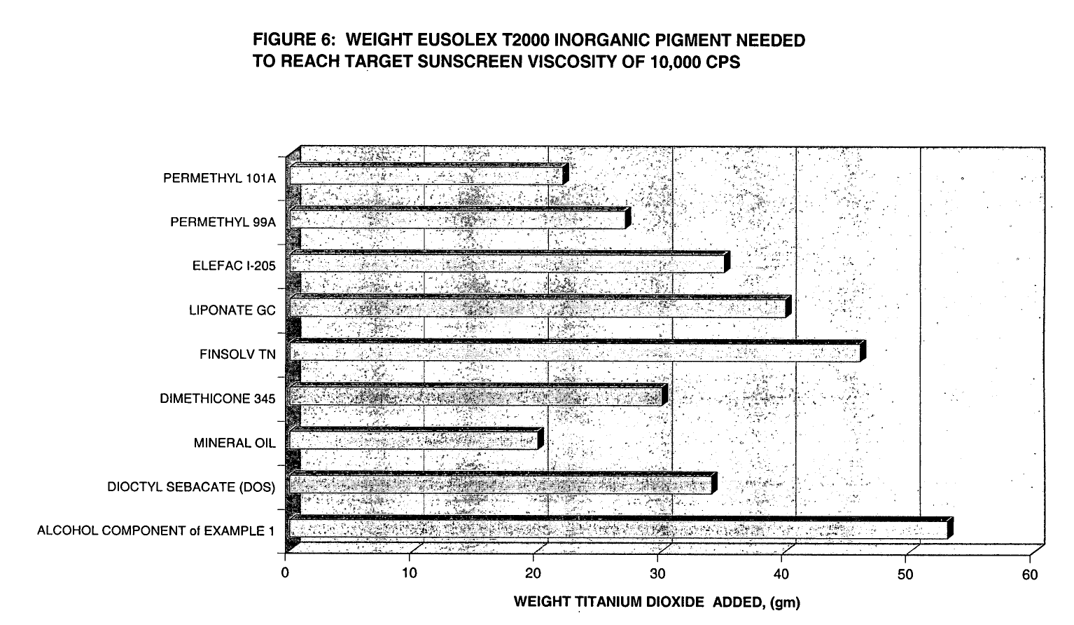

These experiments were further repeated using different types of inorganic sunscreens, namely hydrophobically-modified zinc oxide (commercially available from BASF Corporation, Nutrition and Cosmetics, 3000 Continental Drive North, Mt. Olive, N.J. 07828, USA under the tradename Z-cote HP-1) and titanium dioxide (commercially available from Merck & Co., Inc., Whitehouse Station, N.J., USA, under the tradename Eusolex T2000), in place of Z-cote. Results from the experiments using Z-cote HP1 are shown in FIG. 2, Table 2 and FIG. 5. Results from the experiments using Eusolex T2000 are shown in FIG. 3, Table 3 and FIG. 6.

TABLE 1
Amount of Z-cote sunscreen needed to give a
dispersion having a viscosity of 10,000 cps.

| | Amount in grams of Z-cote |
|---|---|
| | needed to give a viscosity |
| Solubilizer/Dispersant | of 10,000 cps |
| Permethyl 101A¹ (Comparative) | 30 |
| Permethyl 99A² (Comparative) | 34 |
| Elefac I-205³ (Comparative) | 24 |
| Liponate GC⁴ (Comparative) | 60 |
| Finsolv TN⁵ (Comparative) | 34 |
| DC 345⁶ (Comparative) | 32 |
| Mineral Oil (Comparative) | 23 |
| Dioctyl sebacate (DOS) | 20 |
| (Comparative) | |
| Alcohol component prepared | 60 |
| according to Example 1 | |

¹isohexadecane commercially available from Presperse Inc., 635 Pierce St., Somerset, NJ 08873, USA
²isododecane commercially available from Presperse Inc., 635 Pierce St., Somerset, NJ 08873, USA
³octyldodecyl neopentanoate commercially available from Bernel Chemical Company, 174 Grand Avenue, Englewood, NJ 07631, USA
⁴caprylic/capric triglyceride commercially available from Lipo Chemicals Inc., 207 19^{th} Avenue, Paterson, NJ 07504, USA
⁵C12-C15 alkyl benzoate commercially available from Finetex Inc., P.O. Box 216, 418 Falmouth Avenue, Elmwood Park, NJ 07407, USA
⁶Cyclomethicone commercially available from Dow Corning Corporation, P.O. Box 994, 2200 West Salzburg Road, Midland, MI 48686-0994, USA

The results from Table 1 are represented graphically in the bar chart of FIG. 4.

TABLE 2
Amount of Z-cote HP1 sunscreen needed to give a
dispersion having a viscosity of 10,000 cps.

| | Amount in grams of Z-cote |
|---|---|
| | HP1 needed to give a |
| Solubilizer/Dispersant | viscosity of 10,000 cps |
| Permethyl 101A¹ (Comparative) | 85 |
| Permethyl 99A² (Comparative) | 112 |
| Elefac I-205³ (Comparative) | 65 |
| Liponate GC⁴ (Comparative) | 46 |
| Finsolv TN⁵ (Comparative) | 48 |
| DC 345⁶ (Comparative) | 74 |
| Mineral Oil (Comparative) | 49 |
| Dioctyl sebacate (DOS) | 50 |
| (Comparative) | |
| Alcohol component prepared | 60 |
| according to Example 1 | |

The results from Table 2 are represented graphically in the bar chart of FIG. 5.

TABLE 3
Amount of Eusolex T2000 sunscreen needed to give a
dispersion having a viscosity of 10,000 cps.

| | Amount in grams of Eusolex |
|---|---|
| | T2000 needed to give a |
| Solubilizer/Dispersant | viscosity of 10,000 cps |
| Permethyl 101A¹ (Comparative) | 22 |
| Permethyl 99A² (Comparative) | 27 |
| Elefac I-205³ (Comparative) | 35 |
| Liponate GC⁴ (Comparative) | 40 |
| Finsolv TN⁵ (Comparative) | 46 |
| DC 345⁶ (Comparative) | 30 |
| Mineral Oil (Comparative) | 20 |
| Dioctyl sebacate (DOS) | 34 |
| (Comparative) | |
| Alcohol component prepared | 53 |
| according to Example 1 | |

The results from Table 3 are represented graphically in the bar chart of FIG. 6.

The results in Tables 1 to 3 and the associated bar charts in FIGS. 4 to 6 show that the branched alcohol component prepared according to Example 1 allows 60 grams of Z-cote/Z-cote HP-1 or 53 grams of Eusolex T2000 to be dispersed before a viscosity of 10,000 cps is reached.

As may be seen from the results in Table 1 and FIG. 4, the branched alcohol component of Example 1 allows more or equivalent amounts of Z-cote inorganic sunscreen to be dispersed before a viscosity of 10,000 cps is reached than all of the well known sunscreen solubilizers/dispersants used in the experiments herein (namely, Permethyl 101A, Permethyl 99A, Elefac I-205, Liponate GC, Finsolv TN, DC345, Mineral Oil and dioctyl sebacate (DOS)).

As may be seen from Table 2 and FIG. 5 the branched alcohol of Example 1 allows more or equivalent amounts of Z-cote HP1 inorganic sunscreen to be dispersed before a viscosity of 10,000 cps is reached than many well known sunscreen solubilizers/dispersants used in the experiments herein (including Liponate GC, Finsolv TN, mineral oil and DOS).

As may be seen from Table 3 and FIG. 6 the branched alcohol of Example 1 allows more Eusolex T2000 to be dispersed before a viscosity of 10,000 cps is reached than all of the sunscreen solubilizers/dispersants used in the experiments herein (namely, Permethyl 101A, Permethyl 99A, Elefac I-205, Liponate GC, Finsolv TN, DC345, Mineral Oil and DOS).

As may be seen from Tables 1-3 and FIGS. 1-6, the branched alcohol component of Example 1 provides an overall improved inorganic sunscreen solubiliser/dispersant providing good dispersion of a range of inorganic sunscreens while maintaining viscosity at an acceptable level.

### Example 3

A sunscreen composition containing the branched alcohol component of Example 1 is prepared using the following ingredients:

TABLE 4

| Phase | Ingredient | % by weight |
|---|---|---|
| A | Deionized Water | to 100 |
| A | Tetrasodium EDTA | 0.1 |
| A | Glycerin (96%) | 2.5 |
| A | Carbopol 981⁷ (2% solution) | 10 |
| B | Octyl Methoxycinnamate | 7.5 |
| B | Octyl Salicylate | 5 |
| B | Benzophenone-3 | 3.5 |
| B | Cetyl alcohol | 0.5 |
| B | Lipomulse 165⁸ | 2.5 |
| B | Stearic Acid | 2.5 |
| B | Alcohol component prepared | 7.5 |
| | according to Example 1 | |
| B | DC 200-50⁹ | 0.5 |
| C | NaOH (20% solution) | 0.75 |
| D | Germaben II¹⁰ | 1 |

⁷Carbomer commercially available from Noveon Hilton Davis, Inc., 2235 Langdon Farm Road, Cincinnati, OH 45237, USA
⁸Glyceryl Stearate + PEG 100 stearate commercially available from Lipo Chemicals, Inc, 207 19th Avenue Paterson, NJ 07504, USA.
⁹dimethicone commercially available from Dow Corning Corporation, PO Box 994, 2200 West Salzburg Road, Midland, MI 48686-0994, USA
¹⁰liquid preservative system commercially available from ISP Technologies, PO Box 1006, Bound Brook, NJ 08805, USA, consisting of 20% diazolyldinyl urea, 10% methyl paraben, 10% propyl paraben and 60% propylene glycol.

The sunscreen formulation is manufactured as follows. The ingredients of Phase A are combined together at 75° C. The ingredients of Phase B are combined together at 75° C. Phase B is then added to Phase A. Phase C is added to the resulting mixture, followed by cooling of the mixture to 40° C. and adding Phase D. The formulation is ready for packaging at a temperature of 35° C.

A new random mono-methyl branched, high molecular weight, fluid, primary alcohol has been synthesized via the modified OXO process. This alcohol has interesting handling properties relative to oleo fatty alcohols of comparable molecular weights. Its solubility profile was determined in a number of sunscreen and lotion moisturization solubilizers. Additionally, this fatty alcohol acts as an excellent vehicle for dispersing commonly used make-up and sun-care actives. Its pigment dispersability was determined. The surface-active properties of the anionic derivatives of this fatty alcohol and foam stability profile makes it a good alternative for suspending sensorial ingredients in personal cleansing products. The irritation and skin sensitization profile of this new alcohol is comparable to oleo alcohol and derivatives. Therefore, a fluid C1617 mono-methyl branched fatty alcohol with exceptional formulation viscosity-building characteristics and comparable skin irritation and sensitization properties is an excellent choice for a variety of personal care leave-on and cleansing products.

### Example 4

Oleochemical and petrochemical alcohol derivatives are very well suited for use in a variety of personal cleansing products. This work is focused on use of petrochemically derived fatty alcohols, specifically C1617 mono-methyl branched emollient fatty alcohol obtained via modified Oxo process, which has demonstrated inherent handling and formulation properties which supports use in a variety of oil-in-water and water-in-oil skin-care lotions and sun-screen products.

The objective of this work was to evaluate the efficacy of modified Oxo mono-methyl (MO MM) branched C1617 alcohol in skin and sun-care moisturizers and lotions. This work demonstrates the suitability of a MO MM branched C1617 alcohol as an exceptional emollient/moisturizer with desirable handling and formulation properties when compared to oleo linear alcohols of comparable molecular weight. Additionally, this work verifies that MO MM branched C1617 alcohol and its derivatives are not expected to be skin sensitizers based on results obtained from the Human Repeat Insult Patch Test (HRIPT).

Experimental

Solubility: Two emulsion types (W/O-water-in-oil night cream/lotion and O/W-oil-in-water moisturizer lotion) were prepared. Both types of products incorporated commonly used cosmetic ingredients. The C1617 MM MO and the Guerbet C16 alcohols were used at 15% in the W/O lotion and 10% in the moisturizer.

Stability Tests: All W/O and O/W lotions were placed on stability testing for phase and odor stability monitoring for 90 days at 23° C. (room temperature) and 45° C.

Pigment Dispersion: Pigment was added to a 100 gm of emollient sample. The mixture was blended using a Silverson mixer for 15 minutes at 5000 rpm. The viscosity was measured after the sample had equilibrated to room temperature.

Freeze/Thaw testing: Phase stability was observed using approximately 30 ml of a prototype formulation. After preparation, the samples were cooled until solid for 24 hrs. They were allowed to equilibrate to room temperature when the appearance of the formulation was recorded. This process was repeated for three 24 hr. freeze/thaw cycles.

Primary Irritation Potential in Humans: The test protocols involved a 3-PAD design involving 30 subjects. A moisturizer base cream incorporating commonly used cosmetic ingredients was prepared containing 0.5% C1617 MM MO emollient alcohol and administered to 30 subjects. The conditions were occluded, semi-occluded and open and applied in three 24 hour cycles. Sodium Laurylsulfate was selected as a positive control and saline solution as a negative control.

Human Repeat Insult Patch Test (HRIPT) Test Protocols: A moisturizer base cream incorporating commonly used cosmetic ingredients was prepared containing 0.5% C1617 MM MO emollient alcohol and administered to 124 human subjects. The conditions were semi-occluded with nine induction applications followed by challenge.

Results and Discussion

Hydrophobe Structure

Oleochemical alcohols derived from palm kernel, coconut oils and tallow fatty acids have 100% linear alkyl hydrophobes. Ziegler, Conventional- and Modified-Oxo (MO) technologies that yield alcohols from olefins are well documented in the literature. The data in Tables 5 and 6 shows the carbon number distribution for oleo and petrochemically derived alcohols. The C1617 mono-methyl branched alcohol is prepared by Shell Chemical's modified OXO process utilizing patented catalyst technology specific for insertion of a mono-methyl branch in the hydrophobe.

TABLE 5
Carbon Distribution for Oleo Alcohols

| Source | C8 | C10 | C12 | C14 | C16 | C18 | C20 | C14-18 Unsat |
|---|---|---|---|---|---|---|---|---|
| Coconut | 5-9 | 6-10 | 44-52 | 13-19 | 8-11 | 1-3 | 0-0.4 | 5-11 |
| Palm Kernel | 3-5 | 3-7 | 40-52 | 14-17 | 7-9 | 1-3 | 0-1 | 13-22 |
| Tallow | | | 0-0.2 | 2-8 | 24-37 | 14-29 | 1-1.2 | 43-58 |

TABLE 6
Carbon Distribution for Petrochemically-derived
Emollient Alcohols

| | | | | | | | | | | Pour |
|---|---|---|---|---|---|---|---|---|---|---|
| Chemical Name | Supplier | C14 | C15 | C16 | C17 | C18 | C19 | C20 | % Branching | Point, ° C. |
| Mod OXO Mono Methyl | Shell | 1 | 1 | 35 | 60 | 1 | 1 | 0.2 | 95 | −16 |
| C1617 Branched Alcohol | | | | | | | | | | |
| Guerbet C16 Branched | Cognis Care | | | 100 | | | | | 100 | −18 |
| Guerbet C18 Branched | Cognis Care | | | | | 100 | | | 100 | 0 |

The carbon distribution comparison is significant since it shows the petrochemcially derived emollient alcohols are enriched in higher alkyl carbon numbers that provide the lubricity characteristics advantaged relative to oleo oils. Similarly, the pour point temperatures suggest that both the MO MM C1617 and the Guerbet C16 have excellent handling properties, are odorless, stable to oxidation, fluid and pourable at very low temperatures. Additionally, Table 6 shows the relative degree of beta-carbon branching. The MO MM C1617 hydrophobe branching obtained using Shell's SHOP process is compared relative to Guerbet alcohols with C6 and C8 beta-carbon branches.

Solubility

Mod OXO MM C1617 branched alcohol exhibited an excellent solubility profile with a broad range of lipophilic materials including cyclomethicone, unsaturated oleo oils, castor oil and polar, solvents like propylene glycol 75% ethanol when compared relative to the Guerbet C₁₆ branched alcohol in Table 7.

The solubility of MO MM C1617 alcohol in castor oil and mineral oil, suggests its may be formulated in lipsticks and other pigmented makeup products. Additionally, its solubility in cyclomethicone suggests its suitability for sunscreen products that contain significant quantities of inorganic pigments like zinc oxide and titanium dioxide.

TABLE 7
Solubility Profiles for Competitive Branched
Emollient Alcohols

| | M.O.MM Branched | | Guerbet | |
|---|---|---|---|---|
| | C1617 | | C16 Branched | |
| | @ 50° C. | @ 25° C. | @ 50° C. | @ 25° C. |
| Mineral Oil (90%) | + | + | + | + |
| Propylene Glycol (90%) | + | + | + | +/− |
| Isopropylmyristyl | + | + | + | + |
| Alcohol (90%) | | | | |
| Glycerin (96%) | − | − | − | − |
| Castor Oil (90%) | + | + | − | − |
| Sesame Oil (90%) | + | + | + | + |
| Octyldodecanol (90%) | + | + | + | + |
| C12-15 Alkyl | + | + | + | + |
| Benzoate (90%) | | | | |
| Isododecane (90%) | + | + | + | + |
| Isohexadecane (90%) | + | + | + | + |
| Octyl Dimethyl | + | + | + | + |
| Paba (30%) | | | | |
| Octocrylene (30%) | + | + | + | + |
| Dimethicone 245 (90%) | + | + | + | + |
| Dc 200-100-Dimethicone | +/− | +/− | − | − |
| (90%) | | | | |

Chart Key
+: Soluble +/−: Dispersible +/−: Insoluble

Lotions/Night-Cream Emollient Properties

MO C1617 mono-methyl branched primary alcohol was formulated in a water-in-oil night cream and an oil-in-water moisturizer formula as indicated in Tables 8 and 10 respectively. The viscosity and formula properties of the MO methyl-branched primary alcohol were evaluated relative to a branched C₁₈ emollient alcohol and the data is indicated in Tables 9 and 11 respectively.

TABLE 8
Oil-in-Water Moisturizer Formulation

| Phase | Ingredient | % wt |
|---|---|---|
| A | Cetyldimethicone Copolyol | 5.0 |
| A | PEG-30 (dipolyhydroxystearate) | 1.0 |
| A | Par. Hydrogenated Castor Oil | 2.5 |
| A | Octyl Palmitate | 5.0 |
| A | Fatty Alcohol | 15.0 |
| A | Vitamin E Acetate | 0.1 |
| B | Propilene Glycol | 2.5 |
| B | Hydroxyethyl cellulose | 0.8 |
| B | Sodium Chloride | 0.8 |
| C | DMDM Hydantoin | 0.2 |
| D | Water | 69.1 |

TABLE 9
Oil-in-Water Moisturizer Properties

| Fatty Alcohol | | Freeze-Thaw | Oven |
|---|---|---|---|
| Component | Viscosity, cps | (3 cycles) | Stability |
| Mod OXO MM | 10,400 | Pass | Pass |
| Branched C16, 17 | | | |
| Guerbet C-16 | 7,600 | Pass | Pass |

Compared to oleo alcohol emollients, like cetyl/cetearyl alcohols and branched guerbet alcohols typically used as emollients in moisturizers and night-creams, MO C1617 alcohol has exceptional handling and viscosity-building properties. In a water-in-oil emulsion product, the MO C1617 night cream had excellent slip characteristics, good product texture with a non-oily skin after-feel. Similarly, in an oil-in-water moisturizer, the MO C1617 product demonstrated good rub-in characteristics with a good matte skin after-feel.

TABLE 10
Oil-in-Water Moisturizer Formulation

| Phase | Ingredient | % wt |
|---|---|---|
| A | Water | 63.6 |
| A | EDTA | 0.1 |
| A | Glycerine | 2.5 |
| A | Poly acrylic copolymer (2.5%) | 15.0 |
| B | Fatty Alcohol | 10.0 |
| B | Glyceryl Stearate, PEG-100 | 2.5 |
| | Stearate | |
| B | Stearic Acid | 2.5 |
| B | Cetearyl Alcohol | 1.0 |
| B | Dimethicone DC 200-50 | 1.0 |
| C | 20% NaOH | 0.8 |
| D | Propylene Glycol, Diazolidinyl | 1.0 |
| | Urea, Methylparaben, Propylparaben | |

TABLE 11
Oil-in-Water Moisturizer Properties

| Fatty Alcohol | | Freeze-Thaw | Oven |
|---|---|---|---|
| Component | Viscosity, cps | (3 cycles) | Stability |
| Mod OXO MM | 35,600 | Pass | Pass |
| Branched C16, 17 | | | |
| Guerbet C-16 | 11,300 | Pass | Pass |

TABLE 12
Sunscreen Emollients and Pigments

| INCI Name | Supplier |
|---|---|
| Mod OXO Mono-methyl C1617 | Shell |
| Branched Alcohol | |
| Octyldodecyl Neopentanoate | Bernel Chemical Co. |
| Caprylic/Capric Triglyceride | Lipo Chemicals Inc. |
| C12-15 Alkyl Benzoate | Finetex |
| Dioctyl Sebacate | Trivent Chemical Co. |
| Isododecane | Presperse Inc. |
| Isohexadecane | Presperse Inc. |
| Microfine ZnO₂ | BASF |
| Transparent, Microfine ZnO₂, coated | BASF |
| with Triethoxycaprylyl Silane | |
| 99% TiO2 with non-toxic Polyol added | Merck & Co. |
| for Processability | |

Sun-screen Properties Table 12 lists the MO MM C1617 branched alcohol and other competitive emollients and pigments tested.

Sun-screen Emollient Properties

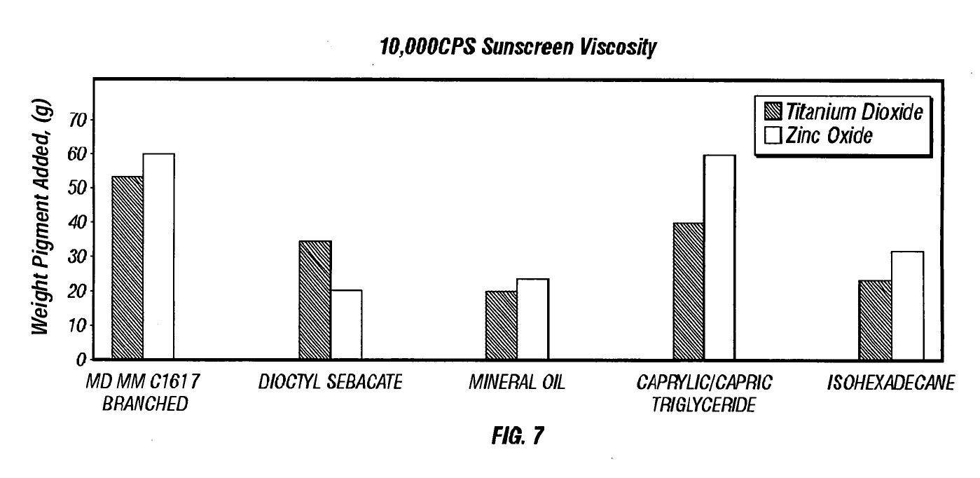

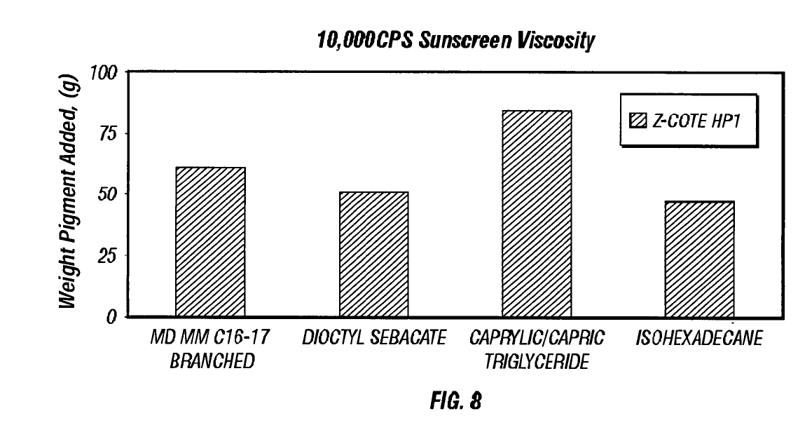

Table 13 lists the sun-screen ingredients. Mod OXO MM C1617 branched alcohol demonstrated excellent capability to create a fine dispersion containing a inorganic sun-screen pigments. Its efficacy to disperse inorganic pigments as shown in FIGS. 5 and 6 and in FIGS. 7 and 8 as compared to other emollients, allows the formulator flexibility to add pigments to boost the SPF factor applied to over-the-counter products to help consumers select products for sun-protection. It is also advantageous that MO MM C1617 has excellent rub-in and good skin afterfeel properties.

TABLE 13
Sunscreen Formulation

| Phase | Ingredient | Wt, % |
|---|---|---|
| A | Water, dionized | 100.0 |
| A | Tetrasodium EDTA | 0.1 |
| A | Glycerin (96%) | 2.5 |
| A | Cross-linked high mol wt acrylic acid polymer | 10.0 |
| | (2% Sol) | |
| B | Octyl Methoxycinnamate | 7.5 |
| B | Octyl Salicylate | 5.0 |
| B | Benzophenone-3 | 3.5 |
| B | C16 Linear Fatty Alcohol | 0.5 |
| B | Glyceryl Stearate & PEG 100 Stearate | 2.5 |
| B | C18 Linear Fatty Acid | 2.5 |
| B | Sun-Screen Emollient | 15.0 |
| B | Dimethicone DC 200-50 | 0.5 |
| C | NaOH (20% soln qs to pH 7.5) | 0.8 |
| D | Preservative | 1.0 |

Skin Irritation and Sensitivity of Mod OXO Mono-methyl C1617 Branched Alcohol

A consumer's perception of skin irritation attributed to personal care products generally involves an itching and/or burning sensation, which may involve dryness and scaling.

Formula additives are included to improve mildness, texture and rub-in afterfeel. Recent data was developed on an oil-in-water moisturizer formulation containing 0.5% w MO MM branched C1617 alcohol using a Human Irritation Potential screening test and for sensitization in a Repeat Insult Patch test (HRIPT). The moisturizer was administered in nine induction applications followed by challenge to one hundred twenty-four test subjects following typical HRIPT protocols.

The results of this test indicated that skin-care lotions or moisturizers formulated with MO MM C1617 branched alcohol are essentially non-irritating and non-sensitizing.

### Example 5

This example compares the alcohol of the present invention to several other dispersing agents in terms of in vitro SPF and viscosity. In vitro SPF means that the test was not carried out on human skin, but an artificial medium. The sunscreen formulation is manufactured as follows. The ingredients of Phase A are combined together at 75° C. The ingredients of Phase B are combined together at 75° C. Phase B is then added to Phase A. Phase C is added to the resulting mixture, followed by cooling of the mixture to 40° C. and adding Phase D. The formulation is ready for packaging at a temperature of 35° C.

TABLE 14
Comparison of Dispersing Agents

| SPF Data | | | | | % by |
|---|---|---|---|---|---|
| Dispersing Agent | In-vitro SPF | No. | Phase | Ingredient | Weight |
| MO C1617 MM Branched | 19.07 | | | | |
| Mineral Oil | 26.3 | | | | |
| C12-15 Alkyl Benzoate | 21.87 | | | | |
| Guerbet C18 Branched | 31.47 | 1 | A | Water, Deionized | 48.86 |
| Guerbet C16 Branched | 22.6 | 2 | A | Tetrasodium EDTA | 0.10 |
| Ethylhexylpalmitate | 22.73 | 3 | A | Glycerin 99% | 2.50 |
| | | 4 | A | Carbopol 981 (2% Soln) | 10.00 |
| | | 5 | B | Octyl Methoxy Cinnamate | 7.50 |
| | | 6 | B | Octyl Salicylate | 5.00 |
| | | 7 | B | Oxybenzone | 3.50 |
| Dispersing Agent | Viscosity, cps | 8 | B | Cetyl Alcohol | 0.50 |
| MO C1617 MM Branched | 5200 | 9 | B | Lipomulse 165 | 2.50 |
| Mineral Oil | 18000 | 10 | B | Stearic Acid | 2.50 |
| C12-15 Alkyl Benzoate | 17000 | 11 | B | Dispersing Agent | 15.00 |
| Guerbet C18 Branched | 3000 | 12 | B | Dimethicone DC 200-50 | 0.50 |
| Guerbet C16 Branched | 2700 | 13 | C | NaOH (20% Soln) qs to pH 7.5 | 0.54 |
| Ethylhexylpalmitate | 13000 | 14 | D | Germaben II | 1.00 |
| | | | | Total | 100.00 |

### CONCLUSIONS

Modified OXO mono-methyl branched C1617 alcohol has excellent handling properties, oxidative and color stability relative to oleo fatty alcohols typically used for emolliency. Its solubility, cost profile, formulation properties and skin texture after-feel are excellent relative to Guerbet branched alcohols of comparable molecular weights. The MO MM C1617 alcohol also demonstrates capability to effectively disperse sunscreen pigments and has been tested to be a non-sensitizer for leave-on products; hence expanding its efficacy for skin-care and sun-care products. The capability of the MO MM C1617 alcohol to solubilize organic pigments is equivalent to the branched fatty alcohol of equivalent molecular weight (Guerbet C16).

1. A personal care composition for topical application to the skin or hair comprising
(a) a branched primary alcohol component, having from 8 to 36 carbon atoms and an average number of branches per molecule of from 0.7 to 3.0, said branching comprising methyl and ethyl branches, and said branched primary alcohol component optionally comprising up to 3 moles of alkylene oxide per mole of alcohol;
(b) at least one sunscreen; and
(c) a cosmetically-acceptable vehicle.

2. The personal care composition of claim 1 wherein the sunscreen is selected from an organic sunscreen, an inorganic sunscreen and mixtures thereof.

3. The personal care composition of claim 1 wherein the organic sunscreen is selected from the group consisting of p-aminobenzoic acid derivatives, anthranilates, benzophenones, camphor derivatives, cinnamic derivatives, dibenzoyl methanes, β,β-diphenylacrylate derivatives, salicylic derivatives, triazine derivatives, benzimidazole derivatives, bis-benzoazolyl derivatives, methylene bis(hydroxyphenylbenzotriazole) compounds, the sunscreen polymers and silicones, and mixtures thereof.

4. The personal care composition of claim 1 wherein the inorganic sunscreen is selected from the group consisting of titanium oxide, zinc oxide, and mixtures thereof.

5. The personal care composition of claim 4 wherein the inorganic sunscreen is hydrophobically modified.

6. The personal care composition of claim 1 wherein the average number of branches per molecule in the branched alcohol component is in the range from 1.0 to 3.0.

7. The personal care composition of claim 1 wherein the average number of branches per molecule in the branched primary alcohol component is in the range from 1.5 to 2.3.

8. The personal care composition of claim 1 wherein the branched primary alcohol component comprises less than 0.5 atom % of quaternary carbon atoms.

9. The personal care composition of claim 1 wherein the branched primary alcohol component contains less than 5% of linear alcohols.

10. The personal care composition of claim 1 wherein in the branched primary alcohol component at least 40% of the number of branches in the alcohol are methyl branches.

11. The personal care composition of claim 1 wherein in the branched alcohol component from 5% to 30% of the number of branches in the alcohol are ethyl branches.

12. The personal care composition of claim 1 wherein the branched primary alcohol component comprises from about 0.01% to about 30% by weight of the composition.

13. The personal care composition of claim 1 wherein the composition is in the form of a water-in-oil or oil-in-water emulsion.
